(19) United States
(12) Patent Application Publication
(10) Pub. No.: US 2025/0221814 A1
(43) Pub. Date: Jul. 10, 2025

(54) A DEVICE FOR SKIN BIOPRINTING AND A METHOD OF MAKING A DEVICE FOR SKIN BIOPRINTING

(71) Applicant: Agency for Science, Technology and Research, Singapore (SG)

(72) Inventors: Weijie Cyrus Beh, Singapore (SG); Wan Ling Wong, Singapore (SG)

(21) Appl. No.: 18/844,447

(22) PCT Filed: Mar. 7, 2023
(86) PCT No.: PCT/SG2023/050139

§ 371 Date: Sep. 6, 2024

(30) Foreign Application Priority Data
Mar. 8, 2022 (SG) 10202202346R

(51) Int. Cl. A61F 2/10; A61M 35/00

(52) U.S. Cl. CPC A61F 2/105; A61M 35/003; A61M 2205/07; A61M 2205/103; A61M 2205/106; A61M 2205/123; A61M 2205/3606; A61M 2205/502; A61M 2207/00

(57) ABSTRACT

There is provided a device for skin bioprinting and a method of making a device for skin bioprinting, the device comprising: a dispenser module configured to actuate and dispense a bioink from a first chamber, and to actuate and dispense a crosslinker from a second chamber: a storage module coupled to the dispenser module, the storage module comprising the first chamber for storing the bioink and the second chamber for storing the crosslinker; a mixing module coupled to the storage module for mixing the bioink and the crosslinker dispensed from the first and second chambers respectively; and a rotatable applicator coupled to the mixing module for rotatably applying a mixture of the bioink and crosslinker from the mixing module onto a surface of a patient in need of skin regeneration.

### TECHNICAL FIELD

The present disclosure relates broadly to a device for skin bioprinting and a method of making a device for skin bioprinting.

### BACKGROUND

Treatment for patients with skin diseases and conditions such as burns may involve transplanting either cultured epithelial autograft sheets, or more commonly, skin grafts. The former requires a relatively large amounts of cells, long culturing time, and the resulting sheets tend to be fragile. The latter results in donor-site morbidity and can be aesthetically unpleasing due to the need to “mesh” the skin by flattening and cutting slits into the skin to achieve coverage over a larger area.

Bioprinters have been demonstrated to be useful tools for printing tissue constructs and may potentially bridge the gap between organ shortage and transplantation needs. In general, bioprinting involves recreating the three-dimensional (3D) structure of a tissue to mimic the native architecture of the extracellular matrix in which cells are suspended. In addition, cells can also be incorporated into these constructs.

However, the bioprinters in the current state-of-the-art tend to be complex and expensive pieces of equipment. For example, some bioprinters may have printing components (e.g., print heads) of a substantial size and consequently may not be suitable for use as handheld devices due to its weight and limited maneuverability. Other forms of printheads may have a straight-edged printhead or a printhead having a flat rectangular surface, which may work well only on relatively flat surfaces but may encounter difficulty dispensing materials at contoured body parts such as the nose, ear and armpits.

Thus, there is a need for a device for skin bioprinting and a method of making a device for skin bioprinting, which seek to address or at least ameliorate one of the above problems.

### SUMMARY

In one aspect, there is provided a device for skin bioprinting comprising: a dispenser module configured to actuate and dispense a bioink from a first chamber, and to actuate and dispense a crosslinker from a second chamber; a storage module coupled to the dispenser module, the storage module comprising the first chamber for storing the bioink and the second chamber for storing the crosslinker; a mixing module coupled to the storage module for mixing the bioink and the crosslinker dispensed from the first and second chambers respectively; and a rotatable applicator coupled to the mixing module for rotatably applying a mixture of the bioink and crosslinker from the mixing module onto a surface of a patient in need of skin regeneration.

In one embodiment, the device further comprises a cooling module configured to contain cooling elements for cooling the bioink, the cooling being to prevent premature gelation of the bioink.

In one embodiment, the cooling module is arranged to be adjacent or at least partially surrounding the mixing module such that when in use, the cooling elements contained therein are capable of cooling the mixture of the bioink and crosslinker.

In one embodiment, the dispenser module comprises a first piston configured to displace within the first chamber to dispense the bioink from the first chamber and a second piston configured to displace within the second chamber and dispense the crosslinker from the second chamber.

In one embodiment, the dispenser module further comprises one or more pneumatic inlets configured to introduce gas for actuating the first and/or second pistons.

In one embodiment, the dispenser module further comprises a plunger coupled to the first and the second pistons.

In one embodiment, the mixing module comprises a static mixer.

In one embodiment, the rotatable applicator comprises a rollerball configured to roll along the surface of the patient in need of skin regeneration.

In one embodiment, the rotatable applicator comprises a cylindrical roller configured to roll along the surface of the patient in need of skin regeneration.

In one embodiment, the device further comprises a control unit configured to control the dispensing of the bioink and the crosslinker from the first and second chambers respectively, to the mixing module.

In one embodiment, the control unit is further configured to control the mixing of the bioink and the crosslinker in the mixing module.

In one embodiment, the device further comprises an activation switch electrically coupled to the control unit for activating and/or deactivating the control unit.

In one embodiment, the device further comprises a pneumatic pump coupled to the one or more pneumatic inlets.

In one embodiment, the device is a handheld device.

In one embodiment, the storage module is detachably coupled to the dispenser assembly.

In one embodiment, the mixing module is detachably coupled to the storage module.

In one embodiment, the first chamber and the second chamber of the storage module are independently removable.

In one embodiment, the cooling module comprises the cooling elements.

In one embodiment, the cooling elements comprise phase change material.

In one aspect, there is provided a method of making a device for skin bioprinting, the method comprising: providing a dispenser module configured to actuate and dispense a bioink from a first chamber, and to actuate and dispense a crosslinker from a second chamber; providing a storage module that is coupled to the dispenser module, the storage module comprising the first chamber for storing the bioink and the second chamber for storing the crosslinker; providing a mixing module that is coupled to the storage module for mixing the bioink and the crosslinker dispensed from the first and second chambers respectively; and providing a rotatable applicator that is coupled to the mixing module for rotatably applying a mixture of the bioink and crosslinker from the mixing module onto a surface of a patient in need of skin regeneration.

### Definitions

The term “biocompatible” as used herein is to be interpreted broadly to refer to the ability of a material to perform its intended function without inducing significant inflammatory response, immunogenicity, or cytotoxicity to native cells, tissues, or organs.

The term “bioinert” as used herein is to be interpreted broadly to refer to a material that does not substantially elicit an immune response from a human or animal when it is disposed within or comes into contact with an in-vivo biological environment.

The term “bioink” as used herein is to be interpreted broadly to refer to a composition comprising a hydrogel for bioprinting or a composition comprising a mixture of cells with a hydrogel for bioprinting.

The term “crosslinker” as used herein is to be interpreted broadly to refer to an agent (e.g., compound, composition) capable of causing gelation of the bioink. For example, the term “crosslinker” may include an agent capable of chemically connecting macromolecules with covalent or ionic bonds. For example, the term “crosslinker” may include an agent capable of altering pH of the bioink. Accordingly, the terms “crosslink” and “crosslinking” as used herein refer to a step of causing gelation of the bioink.

The term “hydrogel” as used herein is to be interpreted broadly to refer to a network of hydrophilic polymers that are cross-linked via covalent or non-covalent bonds. Due to the hydrophilic nature of hydrogel constituents, hydrogels swell by absorbing water in an aqueous solution but do not dissolve because of a crosslinking structure thereof.

The term “dynamic mixer” as used herein is to be interpreted broadly to refer to an in-line mechanical device with at least one movable mixing element designed to provide continuous mixing of fluid (e.g., gas or liquid).

The term “static mixer” as used herein is to be interpreted broadly to refer to an in-line device with at least one fixed/stationary/non-movable (e.g., in a translational manner) mixing element designed to provide continuous mixing of fluid (e.g., gas or liquid). The static mixer may comprise a series of non-movable (e.g., in a translational manner) mixing elements arranged in a housing. The mixing elements may be, for example, helical elements and/or baffles. The housing and the mixing elements of the static mixer may be independently made of metal or plastic. Typical materials for static mixer components may include but are not limited to stainless steel, polypropylene, polyacetal, polytetrafluoroethylene (PTFE), polyvinylidene difluoride (PVDF) and polyvinyl chloride (PVC).

The term “substrate” as used herein is to be interpreted broadly to refer to any supporting structure.

The term “layer” when used to describe a first material is to be interpreted broadly to refer to a first depth of the first material that is distinguishable from a second depth of a second material. The first material of the layer may be present as a continuous film, as discontinuous structures or as a mixture of both. The layer may also be of a substantially uniform depth throughout or varying depths. Accordingly, when the layer is formed by individual structures, the dimensions of each of individual structure may be different. The first material and the second material may be same or different and the first depth and second depth may be same or different.

The term “continuous” when used to describe a film or a layer is to be interpreted broadly to refer to a film or a layer that is substantially without gaps or holes or voids across the film or layer. In this regard, a continuous film or a continuous layer is also intended to include a film or a layer that may have trivial gaps or holes or voids that may not appreciably affect the desired properties of the film or the layer.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a device for skin bioprinting, a method of making a device for skin bioprinting, and a method of applying a mixture of a bioink and crosslinker are disclosed hereinafter.

### Device for Skin Bioprinting

In various embodiments, there is provided a device for skin bioprinting comprising: a dispenser module configured to actuate and dispense a bioink from a first chamber, and to actuate and dispense a crosslinker from a second chamber; a storage module coupled to the dispenser module, the storage module comprising the first chamber for storing the bioink and the second chamber for storing the crosslinker; a mixing module coupled to the storage module for mixing the bioink and the crosslinker dispensed from the first and second chambers respectively; and a rotatable applicator coupled to the mixing module for rotatably applying a mixture of the bioink and crosslinker from the mixing module onto a surface of a patient in need of skin regeneration.

In various embodiments, the device may be a handheld bioprinter that is designed to deposit the mixture of bioink and crosslinker (e.g., cell-laden bioink) directly onto the surface (e.g., wound bed). By coupling a mixing mechanism (to mix a bioink base with crosslinker(s)), to a rotatable applicator (e.g., rollerball attachment), the device may advantageously achieve even deposition of the mixture of bioink and crosslinker onto a contoured surface such as the human body.

In various embodiments, the components of the device (e.g., dispenser module, storage module, mixing module, and rotatable applicator) may all be integral to the device and are not detachable. In various embodiments, one or more components of the device (e.g., dispenser module, storage module, mixing module, and/or rotatable applicator) may be detachable from the device. In various embodiments, one or more the components of the device (e.g., dispenser module, storage module, mixing module, and/or rotatable applicator) may be disposable and replaceable. The modularity of the device may allow the individual components to be replaced independently of one another. This may advantageously minimize cross-contamination when reusing the device, as components that come into contact with biological matter (e.g., the patient's cells) can be disposed and replaced after use. In various embodiments, the simple configuration of the device may contribute to the ease of replaceability of its various components, as compared to more complex bioprinting devices known in the art where the components are manifestly not disposable owing to their complex construction.

In various embodiments, one or more components of the device is/are biocompatible, bioinert, and sterilisable. In various embodiments, parts of the device that come into contact (directly or indirectly) with the surface of the patient is/are biocompatible, bioinert, and sterilisable. In various embodiments, the whole device is biocompatible, bioinert, and sterilisable. Sterilisation may be performed using suitable materials and techniques known in the art, e.g., ethylene oxide, gamma or electron beam irradiation, plasma, or autoclave sterilization. Accordingly, one or more components of the device is/are made of materials that are able to withstand the desired sterilization methods without substantial or appreciable loss in their desired properties or functions. In various embodiments, the device may be substantially devoid of any electronic components. This may advantageously facilitate ease of sterilization as the device may be amenable to a greater selection of sterilization techniques.

In various embodiments, the patient in need of skin regeneration or skin grafting may be a patient suffering from a skin disease or condition. In various embodiments, the patient in need of skin regeneration may have damaged or missing skin that cannot heal on its own. In various embodiments, the skin disease or condition may be a burn, infection, cancer, e.g., skin loss due to surgery to remove skin cancer, skin ulcer, pressure injury, e.g., bedsores, slow healing wound or very large wound, e.g., an open fracture that breaks the skin. In various embodiments, the burn may be a friction burn, cold burn, thermal burn, radiation burn, chemical burn, electrical burn, or a mixture thereof. In various embodiments, the burn may be categorized based on how deeply the skin has been harmed, namely, a first degree burn, a second degree burn, a third degree burn, or a fourth degree burn. Advantageously, the device for skin bioprinting may be capable of depositing a substantially contiguous layer of bioink (e.g., bioink laden with keratinocytes) directly onto the surface (e.g., dermis) of the patient, thereby achieving a better cosmetic outcome for the patient. In various embodiments, the device for skin bioprinting may alternatively be used to apply the mixture onto a tissue culture surface/substrate for in-vitro tissue culture. In various embodiments therefore, the device may be used in applications such as skin bioprinting, wound healing, burns, chronic wound management, fabrication of in vitro tissue models, deposition of non-collagen bioinks such as alginate and calcium salts etc.

### Housing

In various embodiments, the device further comprises a housing/casing. The housing may comprise an external surface for allowing an operator e.g., healthcare professional to hold/grip the device, and a chamber defined within the housing for holding various components of the device, e.g., dispenser module, storage module, mixing module and rotatable applicator. In various embodiments, the housing may be a single integrated housing. In various embodiments, the housing may be a plurality of separate housings for the respective modules, said plurality of separate housings capable of being assembled together to form the device. In various embodiments, the housing may further comprise one or more outlets/openings for allowing various components of the device, e.g., rotatable applicator, to be accessible from outside of the housing or to be installed inside the housing. The various components of the device may be detachable and replaceable. The various components of the device may be arranged to move independently of one another and/or to move in a coordinated manner within the housing.

In various embodiments, the device may be a handheld device, i.e., shaped and sized to be held by a human hand. The housing may have a specific shape and/or form for ergonomic purposes that facilitates manipulation by an operator e.g., a healthcare professional. In various embodiments, the device may comprise one or more controls e.g., electronic controls, physical controls (e.g., button, knob, slider, switch, lever, joystick etc.) disposed on an external surface of the housing. Each of the one or more controls may be associated with actuating a component of the device, e.g., dispenser module. Actuation of a component may comprise controlling the movement, position and/or orientation of the component. Control of actuation may be physical, electronic, manual and/or automatic. The housing of the device may be ergonomically configured to enhance user experience (e.g., ease of fine control, manipulation and handling) and improve comfort during use of the device. The housing of the device may be symmetric, hence allowing for both right and left-handed users. The one or more controls may be disposed on the external surface of the housing in a manner that is easy-to-reach and/or easy to push/press smoothly such that there is minimal or no need for the operator to reposition the holding/gripping position of the device.

### Control Unit

In various embodiments, the device may further comprise a control unit. In various embodiments, the control unit may be configured to control the dispensing of the bioink and the crosslinker from the first and second chambers respectively, to the mixing module. In various embodiments, the control unit may be further configured to control the mixing of the bioink and the crosslinker in the mixing module. In various embodiments, the device may further comprise an activation switch electrically coupled to the control unit for activating and/or deactivating the control unit. In various embodiments, the control unit and/or activation switch may be disposed on the handheld portion (e.g., as a finger trigger) of the device or disposed in the pneumatic pump (e.g., as a foot pedal switch).

### Storage Module

In various embodiments, the storage module functions to hold/accommodate the bioink and crosslinker prior to mixing and dispensing from the device. In various embodiments, the first and second chambers may each take the form of an elongate container having a first end and a second end opposite to the first end. The elongate container may have a suitable cross-sectional shape, e.g., circular, semi-circular, elliptical, semi-elliptical, square, or rectangular shape. The first and second chambers may each comprise at least one opening at the first end and at least one opening at the second end thereof.

In various embodiments, the openings at the respective first ends of the first and second chambers may be configured to be coupled to the dispenser module to facilitate transmission of actuating forces from the dispenser module to the bioink in the first chamber and the crosslinker in the second chamber. In various embodiments, the openings at the respective second ends of the first and second chambers may be configured to be coupled to the mixing module to facilitate dispensing of the bioink from the first chamber and the crosslinker from the second chamber.

In various embodiments, the storage module may be detachably coupled to the dispenser module/assembly. In various embodiments, the storage module may be detachably coupled to the mixing module. In various embodiments, the first chamber and the second chamber of the storage module may be independently removable. In various embodiments, the openings at the first and second ends of the chambers may comprise connectors that facilitate detachable coupling of the first and second chambers with the dispenser module and the mixing module, respectively. In various embodiments, the first chamber and second chamber of the storage module may be disposable and replaceable. In various embodiments, the first chamber and second chamber of the storage module may be in the form of disposable cartridges. Advantageously, this allows fresh cartridges of the bioink and crosslinker to be inserted into the device once the existing cartridges of the bioink and crosslinker are depleted.

### Mixing Module

In various embodiments, the mixing module functions to mix the bioink and the crosslinker dispensed from the first and second chambers respectively. In various embodiments, mixing of the bioink and the crosslinker causes the bioink to undergo gelation. Advantageously, premixing with the mixing module may ensure that the materials (e.g., bioink and crosslinker) are evenly mixed prior to application/dispensing. In addition, premixing with the mixing module may promote better healing and regeneration of the skin surface. In various embodiments, the mixing module may be disposable and replaceable. Advantageously, by having a disposable and replaceable mixing module, cross-contamination can be minimized when reusing the device.

In various embodiments, the mixing module may comprise a mixing chamber, a mixer disposed within the mixing chamber, at least one opening at a first end, and at least one opening at a second end opposite to the first end of the mixing module.

In various embodiments, mixing module may comprise two openings at the first end. In various embodiments, each opening at the first end of the mixing module may be configured to be coupled to the opening at the second ends of the first and second chambers of the storage module to receive the bioink and crosslinker, respectively. In various embodiments, the mixing module may comprise an opening (e.g., nozzle) at the second end configured to dispense the mixture of bioink and crosslinker to the rotatable applicator.

In various embodiments, the mixer may be a combination of a static mixer and a dynamic mixer. In various embodiments, the mixer may be a dynamic mixer only. In various embodiments, the mixer may be a static mixer only. The inventors have recognized that in various embodiments, a mixing module consisting of the static mixer alone may be sufficient for rapidly achieving even mixing of the bioink and crosslinker. The use of a static mixer alone may also advantageously provide a simpler construction of the device by reducing unnecessary bulk (e.g., additional mechanical and electronic components for configuring a dynamic mixer) and allows the device to be handheld.

### Rotatable Applicator

In various embodiments, the rotatable applicator functions to apply the mixture of bioink and crosslinker from the mixing module onto the surface of the patient in need of skin regeneration. In various embodiments, the mixture of bioink and crosslinker is in a gel form when applied onto the surface. In various embodiments, the rotatable applicator may be capable of applying a substantially even layer of the mixture of bioink and crosslinker. In various embodiments, the rotatable applicator may be in direct or indirect contact with the skin surface of the patient when applying the mixture of the bioink and crosslinker. In one example, the rotatable applicator may contact and apply the mixture of the bioink and crosslinker directly on the skin surface of the patient (i.e., direct application). In another example, the rotatable applicator may contact and apply the mixture of the bioink and crosslinker on a dressing (e.g., dermal regeneration template) disposed on the surface of the patient (i.e., indirect application).

In various embodiments, the rotatable applicator may comprise a rollerball configured to roll along the surface of the patient in need of skin regeneration. In various embodiments, the rollerball may be configured to rotate about two or more axes (e.g., 3 axes) of rotation. In various embodiments, the rollerball may facilitate omnidirectional deposition of material, e.g., the mixture of bioink and crosslinker. In various embodiments, the rotatable applicator may further comprise a rollerball adapter for coupling the rollerball to a nozzle of the mixer.

In various embodiments, the rollerball may advantageously allow a relatively large surface area to be covered in a relatively short amount of time as compared to a nozzle for dispensing material, e.g., mixture of bioink and crosslinker. In various embodiments, the rollerball may advantageously allow deposition of material over contoured surfaces (e.g., contoured body surfaces such as nose and ear) more easily. In various embodiments, the use of a rollerball in a bioprinter may be particularly relevant in the context of a handheld device that is capable of being easily manipulated over a contoured surface (e.g., of human anatomy), and is capable of providing the physical contact which is necessary for the rolling action which evenly distributes the mixture of bioink and crosslinker. On the other hand, the inventors have recognized that physical contact with a contoured surface cannot be easily maintained for a printhead which is mounted on an XYZ Cartesian stage (as most bioprinters are), which makes the use of a printhead extraneous and impractical.

In various embodiments, the rotatable applicator may comprise a cylindrical roller configured to roll along the surface of the patient in need of skin regeneration. In various embodiments, the cylindrical roller may be configured to rotate about a single axis of rotation. In various embodiments, the rotatable applicator may further comprise a cylindrical roller adapter for coupling the cylindrical roller to a nozzle of the mixer. In various embodiments, the cylindrical roller adapter may comprise a shaft for allowing the cylindrical roller to be mounted thereon.

In various embodiments, the rollerball adapter and cylindrical roller adapter may comprise one or more hollow compartments for allowing a cooling element to be contained therein, said cooling element to provide chilling to the mixture of bioink and crosslinker. Accordingly, in various embodiments, a cooling module may be disposed within the adapter of the rotatable applicator. As the cooling module may at least partially surround a mixing module, in various embodiments, at least part of the mixing module may also be disposed within the adapter of the rotatable applicator.

In various embodiments, the rollerball and cylindrical roller may be capable of applying the mixture of bioink and crosslinker evenly over the contours of the surface of the patient. In various embodiments, the rollerball and cylindrical roller may be configured in different sizes to suit the working location and surface. In various embodiments, the rollerball may have a diameter falling in the range of from about 7.8 mm to about 30 mm. In various embodiments, the diameter of the rollerball may fall in a range with start and end points selected from the following group of numbers: 7, 8, 9, 10, 11, 12, 13, 14, 15, 16, 17, 18, 19, 20, 21, 22, 23, 24, 25, 26, 27, 28, 29, and 30 mm. In various embodiments therefore, the rotatable applicator may be configured to be detachable from the device to facilitate interchanging of rollerballs and cylindrical rollers of different sizes, such that a rollerball or a cylindrical roller with a size that suits the working location and surface can be attached to the device. For example, on the nose and ear regions, the rollerball or cylindrical roller may have a smaller diameter to facilitate application in such regions.

In various embodiments, the use of a roller ball or cylindrical roller may facilitate easier access to highly contoured surfaces on the human anatomy such as the nose, ear and armpits. Accordingly, the rollerball and cylindrical roller may be advantageous over other forms of printheads, such as a straight-edged printhead or a printhead having a flat rectangular surface, which may work well only on relatively flat surfaces but may encounter difficulty dispensing materials at contoured body parts such as the nose, ear and armpits.

### Dispenser Module

In various embodiments, the dispenser module functions to provide one or more actuating forces for dispensing the bioink and crosslinker from the first and second chamber, respectively. In various embodiments, the dispenser module may be a pneumatically driven dispenser module that has an adjustable pressure to achieve different rates of deposition. In various embodiments, the dispenser module may be configured to dispense the bioink and crosslinker at a controllable flow rate/dispensing rate.

In various embodiments, the dispenser module may comprise a first piston configured to displace/move within the first chamber to dispense the bioink from the first chamber and a second piston configured to displace/move within the second chamber and dispense a crosslinker from the second chamber. In various embodiments, the first and second pistons may be coupled to the first and second chambers of the storage module via the respective openings at the first ends of the first and second chambers. In various embodiments, the dispenser module may further comprise a plunger coupled to the first and the second pistons, said plunger configured to drive the first and second pistons. In various embodiments, the plunger and the pistons may be configured to move in tandem.

In various embodiments, the dispenser module may further comprise one or more pneumatic inlets configured to introduce gas for displacing/moving the plunger and the first and second pistons. In various embodiments, the dispenser module may comprise a single pneumatic inlet. Advantageously, the use of a single pneumatic inlet (as opposed to multiple control cables for the motors and coolant lines in conventional bioprinters) may reduce the size of the device, making it less bulky and easier to handle.

In various embodiments, the one or more pneumatic inlets may be coupled to a pump. In various embodiments, the pump may be a positive displacement pump. In various embodiments, the pump may be a pneumatic pump configured to apply gas or air pressure for displacing/moving the plunger and the pistons. In various embodiments, a pneumatically driven dispenser module may advantageously have minimal instrumentation requirements, i.e., requiring only compressed air for dispensing the bioink and crosslinker.

In various embodiments, the plunger may be configured to move in response to a force exerted thereon by the applied air or gas pressure, which in turn causes the first and second pistons to move in tandem. In various embodiments, the plunger and the pistons may be made of material with a relatively low coefficient of friction, e.g., polytetrafluoroethylene (PTFE), to reduce the force required to displace the plunger and the pistons. This may advantageously facilitate fine control of the flow rates/dispensing rates of the bioink and crosslinker. It will be appreciated that the control of the dispensing rates of the bioink and crosslinker may be achieved by other means, including using motor-driven syringes. However, the inventors recognized that such other means may compromise the ease of manipulation of the device due to an increased size of the device. Therefore, in various embodiments, the components of the device may be selected from a viewpoint of achieving a handheld device that is easily manipulated by an operator.

In various embodiments, the minimum pressure required to move the plunger and the pistons may fall in the range of from about 0.5 psi to about 20 psi. In various embodiments, the minimum pressure required to move the plunger and the pistons may fall in the range with start and end points selected from the following group of numbers: 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, and 20 psi. Advantageously, a lower minimum operating pressure may allow fine-tuning and fine control of the dispensing rate/flow rate of the bioink and crosslinker. It will be appreciated that the range of pressure depends on the configuration of the device. For example, it is possible to increase the operating pressure by changing the plunger friction. In various embodiments, the dispenser module may be configured to dispense the bioink and crosslinker at a flow rate falling in the range of from about 5 ml/min to about 100 ml/min. In various embodiments, the flow rate of the bioink and crosslinker may fall in a range with start and end points selected from the following group of numbers: 5, 10, 15, 20, 25, 30, 35, 40, 45, 50, 55, 60, 65, 70, 75, 80, 85, 90, 95, and 100 ml/min.

In various embodiments, the chambers of the storage module may be changeable to store bioink and crosslinker at different ratios without having to change the pneumatic settings of the pump. This ease of usage may provide greater convenience when fluids with varying mixing ratios are used. In various embodiments, the dispenser module may be configured to provide a predictable flow of the bioink and crosslinker by changing the volume of the storage chamber while keeping the pressure and hence plunger speed the same.

In various embodiments, the dispenser module may be controlled by the control unit. In various embodiments, the dispensing of the bioink and crosslinker from the first and second chambers may be based on user input. In various embodiments, the dispensing of the bioink and crosslinker from the first and second chambers may be activated and/or deactivated based on user input via an activation switch electrically coupled to the control unit. In various embodiments, the control unit and/or activation switch may be disposed on the handheld portion of the device or disposed in the pneumatic pump. In various embodiments, the activation switch may be a finger trigger disposed on the handheld portion of the device. In various embodiments, the activation switch may be a foot pedal switch disposed in the pneumatic pump. In various embodiments, when the activation switch is activated, the bioink and crosslinker are dispensed, mixed, and allowed to be applied/rolled onto the surface. In various embodiments, when the activation switch is deactivated, the bioink and crosslinker are not dispensed and does not leak from the device.

### Cooling Module

In various embodiments, the device may further comprise a cooling module configured to contain cooling elements for cooling the bioink, the cooling being to prevent premature gelation of the bioink. In various embodiments, the cooling module functions to provide temperature control by maintaining the bioink at a desired working temperature. In various embodiments, cooling the bioink may prevent clogging of the mixer in the mixer module. In various embodiments, cooling the bioink may allow the bioink to be usable for a longer period of time, thereby allowing an operator, e.g., healthcare professional, more flexibility and leeway when using the device (e.g., in an operating theater).

In various embodiments, the cooling module is arranged to be adjacent or at least partially surrounding the mixing module such that when in use, the cooling elements contained therein are capable of cooling the mixture of the bioink and crosslinker. In various embodiments, the cooling module may comprise one or more compartments arranged to be adjacent or at least partially surrounding the mixing module, said one or more compartments for containing the cooling elements. In various embodiments, the cooling module comprises the cooling elements. In various embodiments, the cooling elements may comprise phase change material. Examples of phase change materials include but are not limited to hydroxyethyl cellulose, sodium polyacrylate, and vinyl-coated silica gel. In various embodiments, the cooling elements may comprise electrically activated cooling elements (e.g., thermoelectric cooling components such as heat pumps and conductive elements etc.).

In various embodiments, the cooling module is configured to maintain the bioink at a temperature falling in the range of from about 0° C. to about 20° C. In various embodiments, the cooling module is typically kept at a temperature lower than the bioink temperature. In various embodiments, phase change materials with different working temperatures (e.g., −20° C., −10° C., −5° C., 0° C., 5° C., 10° C., etc.) may be used to hold the bioink materials at a suitably low temperature to prevent premature gelation.

In various embodiments, the cooling module may provide a relatively simple technique of cooling the bioink as compared to complicated bioprinters in the art that are driven by multiple motors requiring multiple control cables, chilled with coolant supplied through relatively large coolant lines, and has a pistol-like configuration. For existing bioprinters that are available in the art, the bioprinters tend to have larger footprints and are heavier. Because of the thick coolant lines and cables, it would be cumbersome to operate. The pistol configuration is also unwieldy. On the other hand, various embodiments of the device disclosed herein may be operated with at least one pneumatic line, an optional finger trigger wire, and may be held in a pencil grip, which makes it easy to manipulate. In various embodiments, cooling may be achieved by using a phase change material instead of coolant lines, which further improves its compactness and usability.

### Bioink

In various embodiments, the bioink may be applied for bioprinting. In various embodiments, the bioink may be applied for skin regeneration. In various embodiments, the bioink may comprise natural and/or synthetic materials. In various embodiments, the bioink comprises a polymer component. Examples of the polymer component may include but are not limited to alginate, gelatin, collagen, e.g., denatured collagen, undenatured collagen, polyethylene glycol, and functionalized hyaluronic acid and combinations thereof. In various embodiments, the polymer component may be collagen. Collagen is a main component of tissue extracellular matrix and can be derived from sources such as porcine, bovine, or fish tissue. Typically, collagen can be dissolved in acid. Upon mixing with appropriate amounts of base to neutralize the acid, the collagen forms a gel. This gelation process can be accelerated by incubating at 37° C., or retarded by chilling. Other natural polymers such as alginate may also be used as a bioink for printing with the handheld device.

In various embodiments, the bioink may further comprise cells. In various embodiments, the bioink may comprise one or more types of cells. In various embodiments, the cells may be autologous cells (i.e., patient's own cells). In various embodiments, the cells may be allogeneic cells (i.e., cells from a person other than the patient). In various embodiments, the cells to be used for skin regeneration may include skin cells and stem cells to be differentiated into skin cells. In various embodiments, skin cells may include keratinocytes, melanocytes, Langerhans cells, and Merkel cells. In various embodiments, stem cells refer to cells having differentiation potency and may include fibroblasts, myoblasts, adult stem cells, mesenchymal stem cells, adipose-derived mesenchymal stem cells, bone marrow-derived mesenchymal stem cells, nerve-derived mesenchymal stem cells, placenta-derived mesenchymal stem cells, cord blood stem cells or combinations thereof.

In various embodiments, the bioink may comprise one or more additives that are capable of supporting cell survival and growth. For example, the bioink may comprise culture media, physiological salts, buffers, growth factors (i.e., a substance that is capable of regulating the growth and function of a cell), differentiation factors (i.e., a substance that is capable of inducing differentiation of cells into tissues or other cells) and combinations thereof. For example, growth factors or differentiation factors may include a transformed growth factor (TGF), a vascular endothelial growth factor (VEGF), a fibroblast growth factor (FGF), an epithelial growth factor (EGF), a platelet-derived growth factor (PDGF), a hepatocyte growth factor (HGF), an insulin-like growth factor (IGF), a cytokine, a chemokine, or combinations thereof.

In various embodiments, the bioink may be at an acidic pH, at a neutral pH or at a basic pH. In various embodiments, the bioink may be at a physiological pH of about 7.4. In various embodiments, the bioink may be used for bioprinting at a temperature falling in the range of from about 4° C. to about 37° C. For example, the bioink, e.g., acidified collagen, may be kept chilled in the cartridge, e.g., maintained at about 0° C. to about 8° C., typically on ice, and thereafter mixed and deposited chilled. As the bioink exits and lands on the skin surface, it will warm up to about 37° C. In various embodiments, the bioink may be used for bioprinting at room temperature (e.g., from about 20° C. to about 30° C.). In various embodiments, the bioink may be sterile. In various embodiments, the bioink may be biocompatible. In various embodiments, the bioink may be non-toxic. In various embodiments, the bioink may have a viscosity suitable for dispensing from the first chamber of the storage module. In various embodiments, the bioink may have a viscosity suitable for application by the rotatable applicator onto the surface of the patient.

In various embodiments, the bioink may have a viscosity value falling in the range of from about 113 cP to about 8471 cP. In various embodiments, the viscosity of the bioink may fall in a range with start and end points selected from the following group of numbers: 100, 200, 300, 400, 500, 600, 700, 800, 900, 1000, 1500, 2000, 2500, 3000, 3500, 4000, 4500, 5000, 5500, 6000, 6500, 7000, 7500, 8000, and 8500 cP.

### Crosslinker

In various embodiments, the crosslinker may include calcium chloride, calcium sulfate, calcium carbonate, calcium (Ca²⁺), magnesium (Mg²⁺), a base (e.g., sodium hydroxide), glutaraldehyde, genipin, nordihydroguaiaretic acid, tannic acid, procyanidins, glycosaminoglycan (GAG), 1-ethyl-3-(3-dimethylaminopropyl) carbodiimide hydrochloride (EDC), divinylbenzene (DVB), ethylene glycol dimethacrylate (EGDMA), tetra(ethylene glycol) diacrylate (TEGDA), poly(ethylene glycol) diacrylate (PEGDA), and combination thereof.

In one example, where the bioink is acidified collagen, crosslinking may be triggered by neutralization of the acid. The acidified collagen may be stored in the first chamber of the storage module and a base such as sodium hydroxide may be stored in the second chamber of the storage module. Mixing of the base with the acidified collagen triggers neutralization of the acid and causes the collagen to form a gel.

In another example, where the bioink is alginate, crosslinking may be triggered by mixing with a calcium salt such as calcium chloride solution. The bioink comprising alginate may be stored in the first chamber of the storage module and the calcium chloride solution may be stored in the second chamber of the storage module. Mixing of the alginate with calcium chloride causes crosslinking to occur and the alginate to form a gel.

### Method of Making a Device for Skin Bioprinting

In various embodiments, there is provided a method of making a device for skin bioprinting, the method comprising: providing a dispenser module configured to actuate and dispense a bioink from a first chamber, and to actuate and dispense a crosslinker from a second chamber; providing a storage module that is coupled to the dispenser module, the storage module comprising the first chamber for storing the bioink and the second chamber for storing the crosslinker; providing a mixing module that is coupled to the storage module for mixing the bioink and the crosslinker dispensed from the first and second chambers respectively; and providing a rotatable applicator that is coupled to the mixing module for rotatably applying a mixture of the bioink and crosslinker from the mixing module onto a surface of a patient in need of skin regeneration.

In various embodiments, the method may further comprise providing a cooling module configured to contain cooling elements for cooling the bioink, the cooling being to prevent premature gelation of the bioink. In various embodiments, the method may further comprise arranging the cooling module to be adjacent or at least partially surrounding the mixing module such that when in use, the cooling elements contained therein are capable of cooling the mixture of the bioink and crosslinker.

In various embodiments, the step of providing the dispenser module may comprise providing a first piston configured to displace within the first chamber to dispense the bioink from the first chamber and a second piston configured to displace within the second chamber and dispense the crosslinker from the second chamber. In various embodiments, the step of providing the dispenser module may further comprise providing one or more pneumatic inlets configured to introduce gas for actuating the first and/or second pistons. In various embodiments, the step of providing the dispenser module may further comprise providing a plunger coupled to the first and the second pistons. In various embodiments, the method may further comprise providing a pneumatic pump coupled to the one or more pneumatic inlets.

In various embodiments, the step of providing the rotatable applicator may comprise providing a rollerball configured to roll along the surface of the patient in need of skin regeneration. In various embodiments, the step of providing the rotatable applicator may comprise providing a cylindrical roller configured to roll along the surface of the patient in need of skin regeneration.

In various embodiments, the method may further comprise providing a control unit configured to control the dispensing of the bioink and the crosslinker from the first and second chambers respectively, to the mixing module, and further configured to control the mixing of the bioink and the crosslinker in the mixing module. In various embodiments, the method may further comprise providing an activation switch electrically coupled to the control unit for activating and/or deactivating the control unit.

### Method of Applying a Mixture of a Bioink and Crosslinker

In various embodiments, there is provided a method of applying a mixture of a bioink and crosslinker on a surface, the method comprising providing a storage module with the bioink stored in a first chamber and the crosslinker stored in a second chamber; actuating and dispensing the bioink from the first chamber into a mixing module using a dispenser module, and actuating and dispensing the crosslinker from the second chamber into the mixing module using the dispenser module; mixing the bioink and the crosslinker dispensed from the first and second chambers respectively in the mixing module; and applying the mixture of the bioink and crosslinker using a rotatable applicator onto the surface.

In various embodiments, the surface may be a skin surface of a patient in need of skin regeneration. In various embodiments, the method may be performed at room temperature of from about 20° C. to about 30° C. In various embodiments, the method may further comprise cooling the bioink to prevent premature gelation of the bioink. In various embodiments, the method may further comprise rolling the rotatable applicator along the surface.

### BRIEF DESCRIPTION OF FIGURES

FIG. 1A is a schematic view drawing of a device/bioprinter for skin bioprinting in an example embodiment.

FIG. 1B is a schematic exposed view drawing of the device for skin bioprinting in the example embodiment.

FIG. 2A is a schematic view drawing of a device for skin bioprinting in another example embodiment.

FIG. 2B is a schematic exposed view drawing of the device for skin bioprinting in the example embodiment.

FIG. 3 is a photograph showing static mixing of a simulated bioink base and a simulated crosslinker solution using a static mixer in an example embodiment.

FIG. 4 is a photograph of a plunger, e.g., back plunger in an example embodiment.

FIG. 5 is a photograph of a partially disassembled device/bioprinter for skin bioprinting in an example embodiment.

FIG. 6A is a photograph showing a device/bioprinter for skin bioprinting in yet another example embodiment.

FIG. 6B is a photograph showing the device/bioprinter applying a material onto a surface in the example embodiment.

FIG. 7 is a schematic diagram showing a total gel mix composition for bioprinting in an example embodiment.

FIG. 8A is a photograph of a well-plate containing collagen gel in an example embodiment.

FIG. 8B is a photograph showing a close-up view of a collagen gel in a particular well in the example embodiment.

FIG. 8C is a photograph showing the collagen gel held by a pair of forceps in the example embodiment.

FIG. 9 is a graph showing viscosity profiles of different formulations in an example embodiment.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, chemical, electrical and biological changes may be made without deviating from the scope of the invention. Example embodiments are not necessarily mutually exclusive as some may be combined with one or more embodiments to form new exemplary embodiments. The example embodiments should not be construed as limiting the scope of the disclosure.

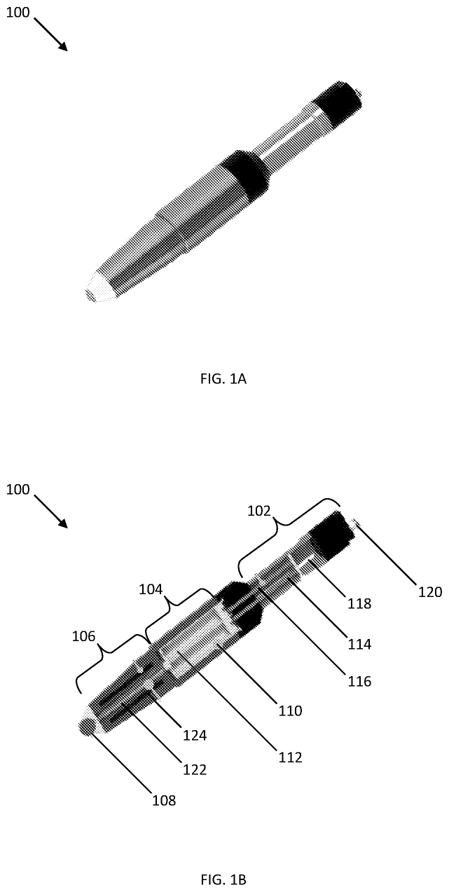

FIG. 1A is a schematic view drawing of a device/bioprinter (100) for skin bioprinting in an example embodiment. FIG. 1B is a schematic exposed view drawing of the device (100) for skin bioprinting in the example embodiment. The device (100) for skin bioprinting comprises a dispenser module (102) configured to actuate and dispense a bioink from a first chamber (110), and to actuate and dispense a crosslinker from a second chamber (112); a storage module (104) coupled to the dispenser module (102), the storage module (104) comprising the first chamber (110) for storing the bioink and the second chamber (112) for storing the crosslinker; a mixing module (106) coupled to the storage module (104) for mixing the bioink and the crosslinker dispensed from the first and second chambers (110, 112) respectively; and a rotatable applicator, e.g., rollerball applicator (108) coupled to the mixing module (106) for rotatably applying a mixture of the bioink and crosslinker from the mixing module (106) onto a surface of a patient in need of skin regeneration.

In the example embodiment, the dispenser module (102) further comprises a first piston (114) configured to displace/move within the first chamber (110) to dispense the bioink from the first chamber (110) and a second piston (116) configured to displace/move within the second chamber (112) and dispense a crosslinker from the second chamber (112). In the example embodiment, the dispenser module (102) further comprises a plunger (118) coupled to the first and the second pistons (114, 116). In the example embodiment, the dispenser module (102) further comprises a pneumatic inlet (120) configured to introduce gas for displacing/moving the plunger (118) and the first and second pistons (114, 116). The pneumatic inlet (120) may be coupled to a positive displacement pump, e.g., pneumatic pump (not shown) configured to apply gas or air pressure for displacing/moving the plunger (118) and the pistons (114, 116). In the example embodiment, the plunger (118) is configured to move in response to a force exerted thereon by the applied air or gas pressure, which in turn causes the first and second pistons (114, 116) to move in tandem. In the example embodiment, the pressure provided to the pneumatically driven dispenser module (102) is adjustable to achieve different rates of deposition. In the example embodiment, the plunger (118) and the pistons (114, 116) may be made of materials with relatively low coefficients of friction, e.g., polytetrafluoroethylene (PTFE), to reduce the force required to displace the plunger (118) and the pistons (114, 116). This may advantageously facilitate fine control of the flow rates/dispensing rates of the bioink and crosslinker.

In the example embodiment, the mixing module (106) comprises a mixing chamber (122) with a mixer, e.g., static mixer disposed within the mixing chamber. The static mixer may comprise a series of non-movable mixing elements, e.g., helical elements and/or baffles, arranged in the mixing chamber (122) to guide the flow and facilitate the mixing of the mixture of bioink and crosslinker travelling from an inlet to an outlet of the mixing module (106). In the example embodiment, the static mixer may achieve rapid mixing of the bioink and the crosslinker.

In the example embodiment, the design of the rollerball applicator (108) allows deposition of material over body contours easily. The use of the rollerball applicator (108) for depositing the mixture of bioink and crosslinker is more relevant in the context of a handheld device, since that allows manipulation of the device over contoured surfaces. The physical contact between the rollerball applicator (108) and the surface for the rolling action aids in spreading the mixture of bioink and crosslinker onto the surface. The inventors have recognized that it is technically difficult to maintain contact with a contoured surface with an XYZ Cartesian stage of a conventional 3D printing instrument, since the instrument must be programmed to adjust the position of its printhead dynamically, which is not easy to accomplish, as most 3D printers using such stages have the tool path pre-loaded, and cannot be changed on-the-fly.

In the example embodiment, the device (100) further comprises a cooling module in the form of a hollow compartment (124) surrounding the mixing module (106). The hollow compartment (124) allows a cooling element, e.g., phase change material, to be contained therein. The cooling element is used to provide cooling/chilling to the mixture of bioink and crosslinker. In order to prevent clogging of the mixer, the bioink, e.g., collagen, should remain chilled both before and after neutralization. This can be achieved using the cooling element, e.g., phase change cooling gel material (e.g., hydroxyethyl cellulose, sodium polyacrylate, or vinyl-coated silica gel), which fill specially designed hollow compartments (124) in the device (100). Before use, the device (100) may be stored refrigerated to cool down the phase change material. Upon deposition onto the surface of the patient, the bioink warms up to the patient's body temperature, and begins to gel.

In the example embodiment, the device (100) is a handheld bioprinter that is designed to deposit bioink, e.g., cell-laden bioink directly onto the surface, e.g., wound bed and contoured surfaces such as the body contours of the patient. The inventors have focused on the usability when designing the device, such that the device can be deployed intraoperatively without being overly cumbersome. Accordingly, in the example embodiment, the device (100) has a relatively small size with minimal instrumentation requirements, relying on compressed air to dispense material. For example, the device (100) may be configured to be operated with only one tubing connected to it (to provide air pressure), as well as a single wire for the finger trigger. The single wire is also optional, since the device (100) can be alternately triggered with a foot pedal switch. This makes the device (100) more compact and convenient to use and manipulate.

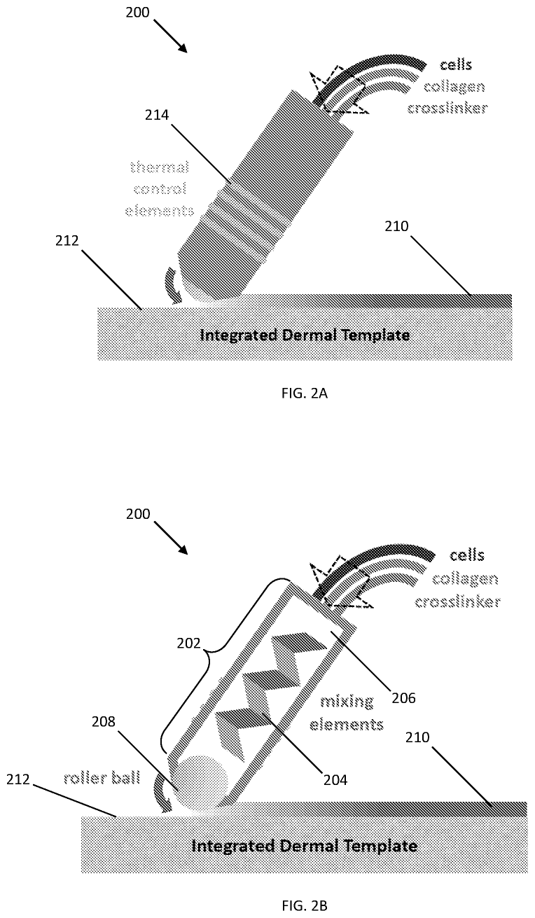

FIG. 2A is a schematic view drawing of a device (200) for skin bioprinting in another example embodiment. FIG. 2B is a schematic exposed view drawing of the device (200) for skin bioprinting in the example embodiment.

In the example embodiment, the device (200) comprises a mixing module (202) comprising mixing elements, e.g., static mixing elements (204) disposed within a mixing chamber (206) and configured for mixing cells, a bioink base, e.g., collagen, and a crosslinker. The device (200) further comprises a rotatable applicator, e.g., rollerball (208) coupled to the mixing module (202) and configured to rotatably apply a mixture (210) of the bioink base, cells, and crosslinker from the mixing module (202) onto a surface (212), e.g., surface of a patient in need of skin regeneration. The device (200) further comprises a cooling module in the form of thermal control elements (214) arranged to be adjacent or at least partially surrounding the mixing module (202) such that when in use, the thermal control elements (214) are capable of cooling the mixture (210) of the cells, bioink and crosslinker.

In the example embodiment, the surface (212) is an integrated dermal template, i.e., a dressing that is placed over a skin surface of a patient. By coupling a mixing mechanism (to mix a bioink base with cells and crosslinker), to a rotatable applicator (e.g., rollerball attachment), the device (200) may advantageously achieve an even deposition of the mixture (210) of cells, bioink and crosslinker onto a contoured surface (212).

In the example embodiment, the thermal control elements (214) help to prevent premature gelation of the bioink, thereby maintaining a longer working time of the bioink, allowing a clinician more flexibility and leeway in the operating theater. In the example embodiment, by coupling a mixing mechanism (to mix a bioink base, cells, and crosslinker) to a rollerball attachment, the mixture can be evenly deposited onto a contoured surface such as the human body. By depositing a contiguous layer of bioink laden with cells, e.g., keratinocytes, directly onto the surface, e.g., dermis, a better cosmetic outcome for the patient may be achieved.

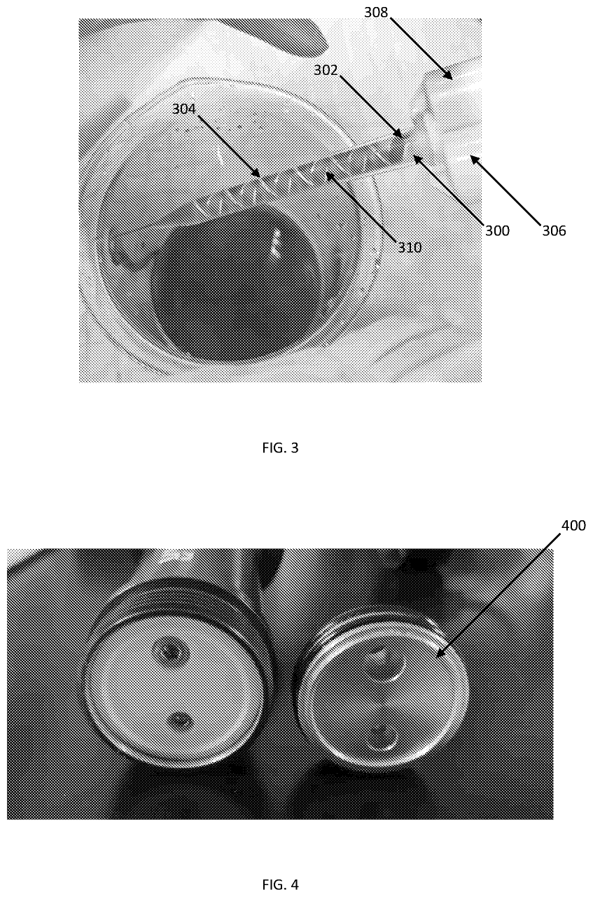

FIG. 3 is a photograph showing static mixing of a simulated bioink base (300) and a simulated crosslinker solution (302) using a static mixer (304) in an example embodiment. In the example embodiment, the simulated bioink base (300) is a colorless solution and the simulated crosslinker solution (302) is a blue colored solution, for ease of visualization. The simulated bioink base (300) and simulated crosslinker solution (302) are simultaneously injected from respective bioink storage chamber (306) and crosslinker storage chamber (308) at one end of the static mixer (304). The mixture of simulated bioink base (300) and simulated crosslinker solution (302) are flowed through a series of baffles (e.g., 310) in the static mixer (304) before exiting via an outlet on the opposite end of the static mixer (304). The experiment demonstrates that the static mixer is capable of achieving rapid and even mixing of the simulated bioink base (300) and the simulated crosslinker solution (302), as evidenced by the homogenous color of the mixture discharged at the outlet of the static mixer (304).

FIG. 4 is a photograph of a plunger, e.g., back plunger (400) in an example embodiment. The back plunger (400) is similar in construction to the plunger (118) of FIG. 1B. Because of the difference in viscosities between the bioink base, e.g., collagen base, and crosslinker, the solutions cannot be directly driven with pressurized air, as the less viscous crosslinker will flow much faster than the bioink base. In the example embodiment, the bioink base and crosslinker are driven using positive displacement mechanisms, e.g., by using pneumatically driven, positive displacement dispensers. Such dispensers may comprise a back plunger (compare 118 of FIG. 1B) that is attached to two pistons (compare 114 and 116 of FIG. 1B).

In the example embodiment, the back plunger (400) is a close-fitting but low friction plunger that is specially fabricated using materials with a relatively low coefficient of friction, e.g., machined from a PTFE block. The back plunger (400) is capable of moving freely, while maintaining a close fit against an inner surface of a dispenser module. In this way, relatively low pressures can be used to drive the pistons (compare 114 and 116 of FIG. 1B), which in turn allows the dispensing rate to be fine-tuned. When attached to a bioink cartridge, the minimum operating pressure needed to actuate the back plunger (400) is from about 2 psi to about 4 psi, which is significantly lower than a back plunger of a conventional positive displacement dispenser.

On the other hand, in a conventional positive displacement dispenser, the back plunger is typically sealed with O-rings, e.g., double O-rings, and does not start moving until around 14-16 psi of pressure is applied. However, since the static friction is higher than the kinetic friction, once the back plunger begins moving, the movement tends to be rapid unless the movement is resisted by the viscous material to be dispensed. Because conventional positive displacement dispensers are designed for high viscosity materials such as two-part epoxy, when used with relatively lower viscosity materials such as collagen and media, the dispensing rate is significantly higher and cannot be easily controlled.

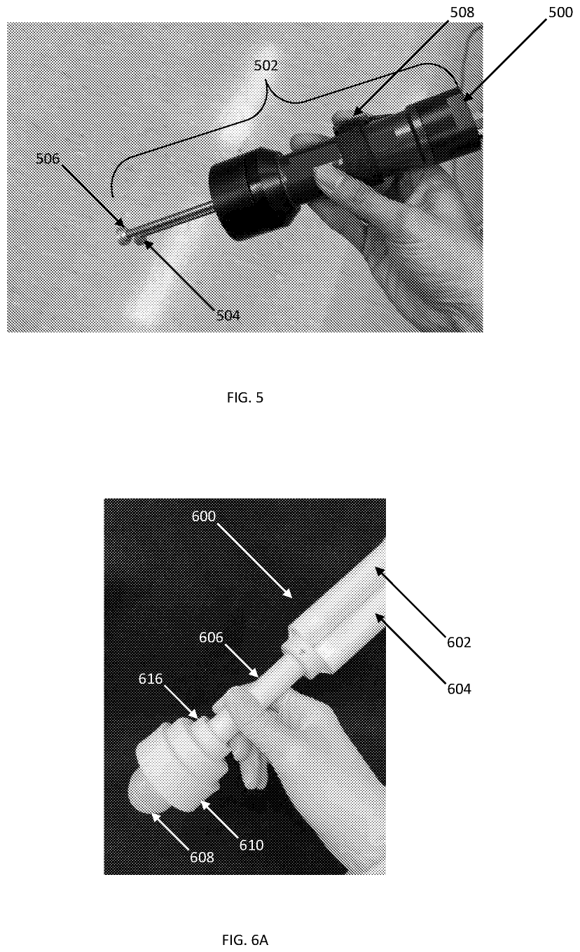

FIG. 5 is a photograph of a partially disassembled device/bioprinter (500) for skin bioprinting in an example embodiment. In the partially disassembled form, the device (500) is shown to comprise a dispenser module (502), said dispenser module (502) comprising a first piston, e.g., pneumatic piston (504) and a second piston, e.g., pneumatic piston (506) (compare 114 and 116 of FIG. 1B). The pneumatic pistons (504, 506) may be coupled to a plunger (not shown) which is driven by a pneumatic pump. The device (500) further comprises an activation switch, e.g., finger-activated trigger (508) disposed on an external surface of the device (500) for easy access by a user's finger. In other words, the dispensing of bioink and crosslinker from the device (500) is based on user input. When the trigger (508) is depressed (i.e., activated), the pneumatic pistons (504, 506) actuate to dispense the bioink and crosslinker, which are then mixed, and applied/rolled onto a surface. When the trigger (508) is released (i.e., deactivated), the bioink and crosslinker are not dispensed and does not leak from the device (500).

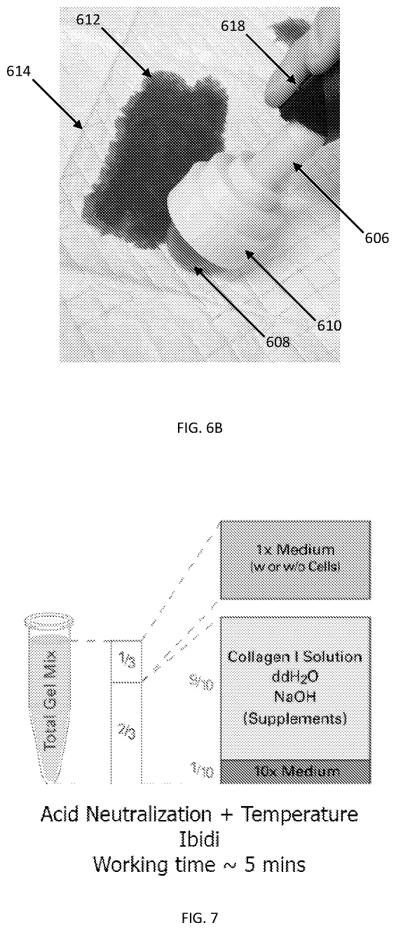

FIG. 6A is a photograph showing a device/bioprinter (600) for skin bioprinting in yet another example embodiment. FIG. 6B is a photograph showing the device/bioprinter (600) applying a material onto a surface (614) in the example embodiment.

In the example embodiment, the device (600) comprises a first cartridge (602) for storing a bioink and a second cartridge (604) for storing a crosslinker. The device (600) further comprises a mixing module (606) for mixing the bioink and crosslinker dispensed from the first cartridge (602) and second cartridge (604). The device (600) further comprises a rotatable applicator, e.g., rollerball (608) for applying a mixture of the bioink and crosslinker (612) dispensed from the mixing module (606). The rollerball (608) is attached to the mixing module (606) via a rollerball attachment interface (610) configured to interface a nozzle of the mixing module (606) to the rollerball (608) and to allow the mixed bioink (612) to be evenly and quickly deposited on the surface (614). The device (600) further comprises a cooling module (616) comprising hollow compartments for containing cooling elements for cooling the mixture of bioink and crosslinker (612). The device (600) further comprises an activation switch, e.g., finger trigger (618) disposed on an external surface of the device (600).

In the example embodiment, the device (600) is a 3D printed prototype fabricated out of polylactic acid (PLA) plastic. The device (600) represents a simplified version of the device (100) of FIG. 1A and FIG. 1B. As the plastic is not a good conductor of heat, the cooling/chilling elements is not included into the cooling module (616). It will be appreciated that the device may be fabricated using other types of material such as aluminum. It will also be appreciated that the prototype may be improvised with better ergonomics for a better hand grip.

During operation, a pneumatic pump coupled to a dispenser module (compare 102 of FIG. 1B) is set to operate at 4 psi and is activated by the finger trigger (618). Upon activation, the bioink and crosslinker are dispensed from the respective first and second cartridges (602, 604) into the mixing module (606), and thereafter the mixture of bioink and crosslinker (612) is dispensed from the nozzle of the mixing module (606) to the rollerball applicator (608). The rollerball applicator (608) is contacted with and rolled along the surface (614). The rolling action of the rollerball applicator evenly distributes the mixture of bioink and crosslinker (612) onto the surface (614). Upon deactivation or release of the finger trigger (618), the dispensing of the bioink and crosslinker stops, and the device (600) does not leak. In the example embodiment, the device (600) was able to apply the mixture of bioink and crosslinker (612) over an area of about 100 cm² within about 10 seconds.

FIG. 7 is a schematic diagram showing a total gel mix composition for bioprinting in an example embodiment. One of three parts by volume of the total gel mix is made up of 1× medium (with or without cells). The other two of three parts by volume of the total gel mix are made up of 10% by volume of 10× medium and 90% collagen I solution, double distilled water, and sodium hydroxide.

In addition to nutrient-rich media and cells, the bioink contains a base material, and a crosslinker. Upon mixing, the bioink forms a hydrogel after some time. Depending on the base material, this gelation can take place nearly instantly (e.g., alginate plus calcium chloride crosslinker solution), or after around 15 mins (e.g., 0.15% neutralized collagen at 37° C.).

Collagen is a main component of tissue extracellular matrix, and can be derived from porcine, bovine, or fish tissue. Typically, collagen can be dissolved in acid. Upon mixing with appropriate amounts of base to neutralize the acid, the collagen forms a gel. This gelation process can be accelerated by incubating at 37° C., or retarded by chilling. Table 1 below shows the time taken for fish collagen of different concentrations to form gel. Based on the time taken for gelation to occur, sufficient time should be allocated for all media/cells to be prepared before it reaches patients.

TABLE 1
Setting time for fish collagen of different concentrations.

| % Fish | | | | |
|---|---|---|---|---|
| Collagen | 0.5 | 0.4 | 0.2 | 0.15 |
| Time to set | 10 | 10 | 12.5 | 15 |
| (mins) | | | | |

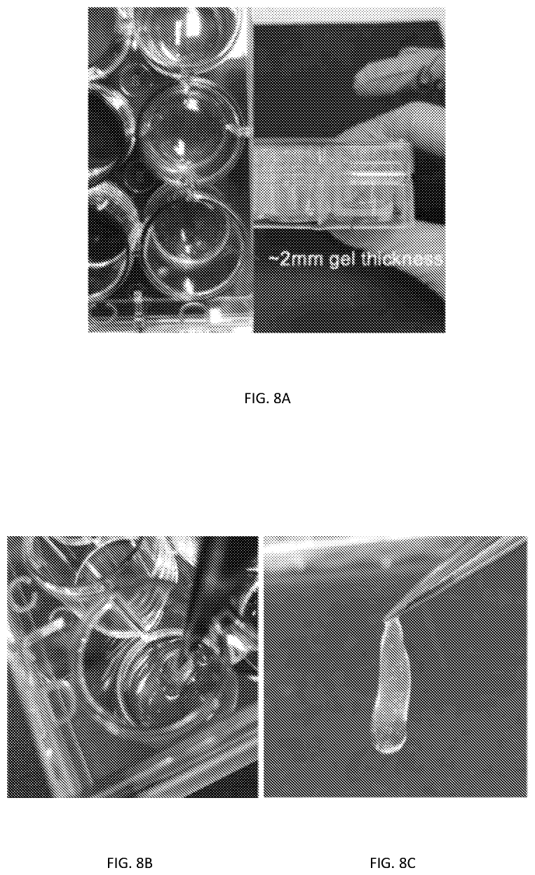

FIG. 8A is a photograph of a well-plate containing collagen gel in an example embodiment. FIG. 8B is a photograph showing a close-up view of a collagen gel in a particular well in the example embodiment. FIG. 8C is a photograph showing the collagen gel held by a pair of forceps in the example embodiment.

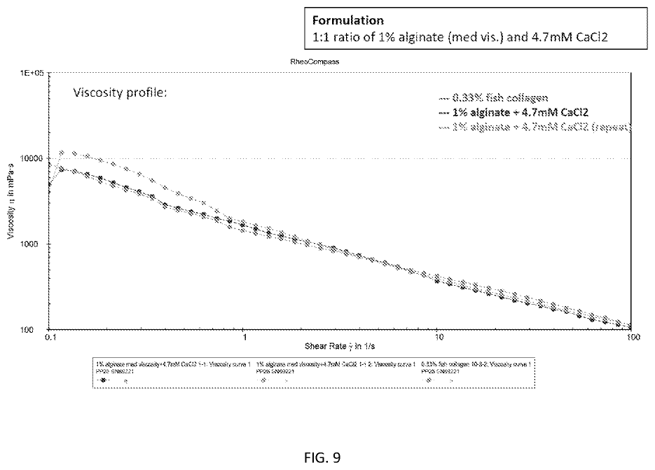

FIG. 9 is a graph showing viscosity profiles of different formulations in an example embodiment. Alginate has a similar viscosity profile as 0.33% fish collagen and therefore can be used as a substitute for fish collagen when testing the device for skin bioprinting as disclosed herein.

### APPLICATIONS

Embodiments of the disclosure provided herein provide a device for skin bioprinting, a method of making a device for skin bioprinting, and a method of applying a mixture of a bioink and crosslinker. Embodiments of the device for bioprinting comprises a dispenser module, a storage module, a mixing module, a rotatable applicator, and optionally a cooling module.

Advantageously, the modularity of the device may allow the individual components to be replaced independently of one another. This may advantageously minimize cross-contamination when reusing the device, as components that come into contact with biological matter (e.g., the patient's cells) can be disposed and replaced after use. In addition, the relatively simple configuration of the device may contribute to the ease of replaceability of its various components, as compared to more complex bioprinting devices known in the art where the components are manifestly not disposable owing to their complex construction.

Advantageously, the device is a handheld device capable of being easily manipulated over a contoured surface (e.g., of human anatomy). In particular, the use of a pneumatic inlet (as opposed to multiple control cables for the motors and coolant lines in conventional bioprinters) may contribute to a reduction in the size of the device, making it less bulky and easier to handle.

Even more advantageously, the device for skin bioprinting may be capable of depositing a substantially contiguous layer of bioink (e.g., bioink laden with keratinocytes) directly onto the surface (e.g., dermis) of the patient, thereby achieving a better cosmetic outcome for the patient. In particular, the use of a rollerball may advantageously allow a relatively large surface area to be covered in a relatively short amount of time as compared to a nozzle for dispensing material, e.g., mixture of bioink and crosslinker. The rollerball may advantageously allow deposition of material over contoured surfaces (e.g., contoured body surfaces such as nose and ear) more easily.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A device for skin bioprinting comprising:
a dispenser module configured to actuate and dispense a bioink from a first chamber, and to actuate and dispense a crosslinker from a second chamber;
a storage module coupled to the dispenser module, the storage module comprising the first chamber for storing the bioink and the second chamber for storing the crosslinker;
a mixing module coupled to the storage module for mixing the bioink and the crosslinker dispensed from the first and second chambers respectively; and
a rotatable applicator coupled to the mixing module for rotatably applying a mixture of the bioink and crosslinker from the mixing module onto a surface of a patient in need of skin regeneration.

2. The device of claim 1, further comprising a cooling module configured to contain cooling elements for cooling the bioink, the cooling being to prevent premature gelation of the bioink.

3. The device of claim 2, wherein the cooling module is arranged to be adjacent or at least partially surrounding the mixing module such that when in use, the cooling elements contained therein are capable of cooling the mixture of the bioink and crosslinker.

4. The device of claim 1, wherein the dispenser module comprises a first piston configured to displace within the first chamber to dispense the bioink from the first chamber and a second piston configured to displace within the second chamber and dispense the crosslinker from the second chamber.

5. The device of claim 4, wherein the dispenser module further comprises one or more pneumatic inlets configured to introduce gas for actuating the first and/or second pistons.

6. The device of claim 4, wherein the dispenser module further comprises a plunger coupled to the first and the second pistons.

7. The device of claim 1, wherein the mixing module comprises a static mixer.

8. The device of claim 1, wherein the rotatable applicator comprises a rollerball configured to roll along the surface of the patient in need of skin regeneration.

9. The device of claim 1, wherein the rotatable applicator comprises a cylindrical roller configured to roll along the surface of the patient in need of skin regeneration.

10. The device of claim 1, further comprising a control unit configured to control the dispensing of the bioink and the crosslinker from the first and second chambers respectively, to the mixing module.

11. The device of claim 10, wherein the control unit is further configured to control the mixing of the bioink and the crosslinker in the mixing module.

12. The device of claim 10, wherein the device further comprises an activation switch electrically coupled to the control unit for activating and/or deactivating the control unit.

13. The device of claim 5, wherein the device further comprises a pneumatic pump coupled to the one or more pneumatic inlets.

14. The device of claim 1, wherein the device is a handheld device.

15. The device of claim 1, wherein the storage module is detachably coupled to the dispenser assembly.

16. The device of claim 1, wherein the mixing module is detachably coupled to the storage module.

17. The device of claim 1, wherein the first chamber and the second chamber of the storage module are independently removable.

18. The device of claim 2, wherein the cooling module comprises the cooling elements.

19. The device of claim 18, wherein the cooling elements comprise phase change material.

20. A method of making a device for skin bioprinting, the method comprising:
providing a dispenser module configured to actuate and dispense a bioink from a first chamber, and to actuate and dispense a crosslinker from a second chamber;
providing a storage module that is coupled to the dispenser module, the storage module comprising the first chamber for storing the bioink and the second chamber for storing the crosslinker;
providing a mixing module that is coupled to the storage module for mixing the bioink and the crosslinker dispensed from the first and second chambers respectively; and
providing a rotatable applicator that is coupled to the mixing module for rotatably applying a mixture of the bioink and crosslinker from the mixing module onto a surface of a patient in need of skin regeneration.
